(19) United States
(12) Patent Application Publication
(10) Pub. No.: US 2026/0218252 A1
(43) Pub. Date: Jul. 30, 2026

(54) IMPROVED ENZYME-ASSISTED SYNTHESIS OF CHLORHEXIDINE BASE

(71) Applicant: Xttrium Laboratories Inc., Mt. Prospect, IL (US)

(72) Inventors: Prajakta Dandekar Jain, Mumbai (IN); Ratnesh Jain, Mumbai (IN); Lalit Khare, East Mumbai (IN)

(21) Appl. No.: 19/148,944

(22) PCT Filed: Jan. 18, 2023
(86) PCT No.: PCT/US23/60826

§ 371 Date: Jul. 17, 2025

(51) Int. Cl. C12P 13/00; C07C 277/08; C07C 279/26; C12N 9/08

(52) U.S. Cl. CPC C12P 13/001; C07C 277/08; C07C 279/265; C12N 9/0065; C12Y 111/0101

(57) ABSTRACT

The present disclosure provides a method for preparing chlorhexidine, salts thereof, or hydrates thereof, wherein the method comprises an enzymatic chlorination, a coupling step, or a combination thereof. The disclosure further includes compositions comprising chlorhexidine prepared by the disclosed methods.

### TECHNICAL FIELD

The present invention relates to methods of preparing chlorohexidine, it's pharmaceutically acceptable salts, and hydrates thereof.

### BACKGROUND

In recent years there has been a dramatic increase in the prevalence of antibiotic-resistant microorganisms. There is considerable concern in the medical community about penicillin resistant pneumococci, vancomycin resistant enterococci and methicillin and quinolone resistant Pseudomonas aeruginosa. Since the early 1980's in the United States and Europe, the incidence of infection by methicillin resistant Staphylococcus aureus (MRSA) and the vancomycin resistant enterococci (VRE) have increased substantially. In medical intensive care units around the country, increased colonization of vancomycin resistant enterococcus has recently reached epidemic proportions. These microorganisms survive by the colonization of the natural environment and new patients through cross contamination. Diarrhea and fecal incontinence further increase the risk of skin colonization by VRE, suggesting that skin colonization is associated with increased risk of catheter related sepsis, cross-infection, and blood culture contamination.

The pathogens isolated from infections differ, primarily depending upon the type of surgical procedure. In clean surgical procedures, in which the gastrointestinal, gynecological, and respiratory tracts have not been entered, Staphylococcus aureus from the exogenous environment or the patient's skin flora is the usual source of infection. According to data from the National Nosocomial Infections Surveillance System, there has been little change in the incidence and distribution of the pathogens isolated from infections in the last decade. However, more of these pathogens show antimicrobial drug resistance, especially methicillin-resistant Staphylococcus aureus.

Wound site infections are a major source of post-operative illness, accounting for many nosocomial infections. These infections number approximately 500,000 per year, among an estimated 27 million surgical procedures, infections that result in longer hospitalizations and higher costs. The average surgical site infection (SSI) prolongs the hospital stay by 7.3 days. These infections contribute to 42% of the extra charges attributed to nosocomial infections. In 1992, the extra costs were estimated at several thousand dollars per SSI infection.

On average, patients with surgical site infections incurred 4.6% extra ambulatory care visits than the patients who did not acquire these infections. In these hospitals, 0.62% to 1.9% of patients with surgical site infections died. These numbers highlight the tremendous human and financial costs that surgical site infections add to the healthcare system and therefore the importance of controlling them.

The antimicrobial effects of bisbiguanides have long been known. The compound 1,6-bis(4-chloro-phenylbiguanido)hexane, also referred to as chlorhexidine, is the best known member of the class and has been used in the form of an aqueous solution or alcohol solution. Chlorhexidine has been marketed for many years in various formulas such as antibacterial hand washes and surgical scrub compositions for disinfection of hands and skin, disinfection of operation site, disinfection of medical instruments, disinfection of wounds, disinfection of operation room, patient's room, and the like. Chlorhexidine gluconate is also commonly known as chlorhexidine digluconate, a salt formed from chlorhexidine and gluconic acid. Chemically, chlorhexidine is a strong base and is most stable in its salt forms.

The structural formula of chlorhexidine is:

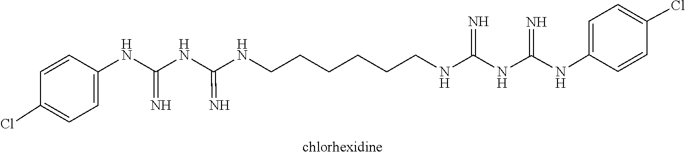 [SMILES: N=C(NCCCCCCNC(=N)NC(=N)Nc1ccc(Cl)cc1)NC(=N)Nc1ccc(Cl)cc1]

Chlorhexidine salts are readily adsorbed onto the cell walls of microorganisms, resulting in disruption of the cell wall integrity and leakage of intracellular contents. At low concentrations, chlorhexidine is a bacteriostatic agent, and at higher concentrations it becomes bacteriocidal. A primary benefit of chlorhexidine is its ability to kill bacteria on contact and remain non-toxic to mammalian cells.

Chlorhexidine is considered the most persistent of all antimicrobial agents currently available. An important attribute of chlorhexidine is its strong affinity for skin. Most of the chlorhexidine gluconate applied to the surface of the skin attaches and remains, accounting for its ability to retain activity upon drying.

Syntheses of chlorhexidine have been previously reported. U.S. Pat. Nos. 2,684,924 and 2,863,919 each disclose the preparation of chlorhexidine as a dihydrochloride salt, where chlorhexidine is prepared by reaction of an amine with a cyanamide adduct. The processes disclosed in both patents, as well as BR9300129, include reacting hexamethylene bis-dicyandiamide with p-chloroaniline (PCA) or reacting hexamethylenediamine with p-chlorophenyldicyanamide. The steps preceding the preparation of p-chlorophenyldicyanamide typically use PCA.

Andrejus Korolkovas, Essentials of Medicinal Chemistry discloses chlorhexidine dichloride using dicyanamide with 1,6-hexanediamine. Hexamethylene-1,6-dicyandiamide is formed as an intermediate and treated with p-chloroaniline hydrochloride leading directly to chlorhexidine dihydrochloride, which is then converted to gluconate salt.

U.S. Pat. No. 4,022,962 discloses a method of preparing chlorhexidine involving the reaction of 1,6-bis(guanidino) hexane sulfate with p-chlorophenyl isocyanate or reaction of hexamethylene diisocyanate with p-chlorophenyl guanidine. Both p-chlorophenyl isocyanate and p-chlorophenyl guanidine are disclosed by U.S. Pat. No. 4,022,962 as being prepared from PCA.

PCA is a toxic substance which shows potential genotoxic properties and may have mutagenic and carcinogenic potential. The presence of PCA in chlorhexidine salts, and hence in chlorhexidine base, is currently restricted by the European Pharmacopoeia to a limit of 500 ppm (i.e. 0.05%) and its use in pharmaceuticals needs to be controlled in order to fulfill the general acceptance of not more than 0.05%. As a result, several methods of purifying chlorhexidine have appeared in order to remove PCA.

BR Patent Application No. PI 9300129A discloses a process for preparing chlorhexidine dihydrochloride similar to those above, where the obtained product contains p-chloroaniline at about 2000 ppm. Several purification procedures are presented where the amount of p-chloroaniline can be reduced to about 500 ppm.

US Patent Publication No. 2013/0065965 is drawn to a process of purifying chlorohexidine to remove PCA by measuring the concentration by HPLC, and if PCA is present in certain amounts, carrying out a purifications process involving suspending chlorhexidine in a solvent and isolating the chlorhexidine from the suspension. US Patent Publication No. 2013/00659 also discloses many impurities found in chlorhexidine compositions which are prone to decompose, resulting in formation of PCA, and that these impurities are less stable in this regard than chlorhexidine itself. As with previous syntheses, the chlorhexidine is disclosed as being prepared by using PCA starting material, and the claims are drawn to reducing PCA to levels of 50-40 ppm or less.

Revelle et al. “Identification and Isolation of Chlorhexidine Digluconate Impurities” Pharmaceutical Research 1993, 10 (12), pp. 1777-1784; and Revelle et al. “Synthesis of Chlorhexidine Digluconate Impurities” J. Agric. Food Chem. 1995, 43, pp. 1299-1301; disclose and characterize impurities from chlorhexidine prepared by older methods such as those mentioned above.

Chloroperoxidase (CPO) is an enzyme that can utilize chloride, bromide and iodide ions as donors for enzymatic halogenation reactions, and can also catalyze classical peroxidation reactions that are characteristic of peroxidases. CPO is thought to be responsible for the chlorination of many natural compounds, and has also been applied to synthetic compounds. For example, Malnar, al., et “Chloroperoxidase-catalyzed chlorination of didechloroaglucovancomycin and vancomycin” Journal of Molecular Catalysis B: Enzymatic, 2000, 10 (6), pp. 545-549 reports the chlorination of vancomycin and related compounds. Malnar also discloses that CPO did not chlorinate all desired positions or compounds, and serves as a demonstration that such a chlorination process can be unpredictable.

PCA, hexamethylenediamine, and dicyanamide are common starting materials used in the production of chlorhexidine. PCA is a known strong carcinogen and toxin. Meanwhile, dicyanamide has significant toxicity to the eyes and lungs. Methods have appeared which directed to reducing PCA in chlorhexidine removal of PCA present from the synthetic route. However, there remains a need for a chlorhexidine synthesis that does not include PCA and dicyanamide as starting materials.

### SUMMARY

The present disclosure provides an improved method of preparing chlorhexidine, a pharmaceutically acceptable salt thereof, or hydrate thereof, comprising an enzymatic chlorination step in combination with one or more steps. The present disclosure also avoids the use of PCA, hexamethylenediamine, and dicyanamide starting materials.

In an embodiment, a method of preparing the compound of formula (II),

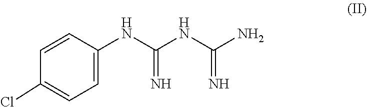 [SMILES: N=C(N)NC(=N)Nc1ccc(Cl)cc1]

a salt thereof, or hydrate thereof comprises enzymatic chlorination of formula (I):

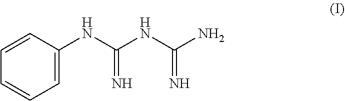 [SMILES: N=C(N)NC(=N)Nc1ccccc1]

or a salt thereof; wherein the chlorination is carried out with chloroperoxidase (CPO), at least one chloride donor, at least one oxidant, and a buffer solution.

In an embodiment, the at least one chloride donor is selected from hydrogen chloride, potassium chloride, sodium chloride, lithium chloride, sodium hypochlorite, calcium hypochlorite, chlorine water, chlorine gas, chloramines, chlorine dioxide, or combinations thereof.

In an embodiment, the at least one chloride donor is sodium chloride.

In an embodiment, the at least one oxidant is selected from hydrogen peroxide, dibenzoyl peroxide, peroxycarboxylic acid, calcium peroxide, lithium peroxide or combinations there.

In an embodiment, the at least one oxidant is hydrogen peroxide.

In an embodiment, the buffer is a phosphate buffer.

In an embodiment, the pH is from about 2.7.

In an embodiment, formula (II) is produced in greater than about a 50% yield.

In an embodiment, a method of preparing chlorhexidine, a salt thereof, or a hydrate thereof, comprises combining a compound of formula (II):

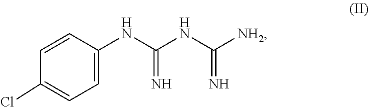 [SMILES: N=C(N)NC(=N)Nc1ccc(Cl)cc1]

a salt thereof, or a hydrate thereof, with a compound of formula (IV):

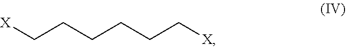

in further combination with one or more base, and wherein X is a leaving group.

In an embodiment, formula (II) is the hydrochloride salt.

In an embodiment, X is selected from hydroxyl, bromine, chlorine, iodine, tosylate, mesylate, triflate, nosylate, or substituted ammonium.

In an embodiment, X is bromine.

In an embodiment, the one or more base is selected from sodium hydroxide, sodium methoxide, sodium ethoxide, potassium hydroxide, potassium tert-butoxide, triethylamine, ethyldiisopropylamine, 1,8-diazabicyclo[5.4.0]undec-7-ene, 7-methyl-1,5,7-triazabicyclo(4.4.0)dec-5-ene, 1,1,3,3-tetramethylguanidine, collidine, 2,6-lutidine, or combinations thereof.

In an embodiment, the one or more base is sodium hydroxide.

In an embodiment, the reaction is conducted in a solvent, the solvent comprising butyronitrile.

In an embodiment, the reaction is carried out at about 100° C.

In an embodiment, the yield of chlorhexidine is greater than about 50%.

In an embodiment, a method of preparing chlorhexidine comprising, the enzymatic chlorination of formula (I):

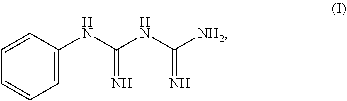 [SMILES: N=C(N)NC(=N)Nc1ccccc1]

or a salt thereof, to form formula (II):

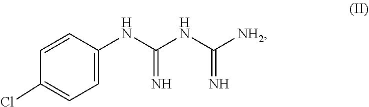 [SMILES: N=C(N)NC(=N)Nc1ccc(Cl)cc1]

a salt thereof, or a hydrate thereof;
the enzymatic chlorination comprising chloroperoxidase (CPO), at least one chloride donor, at least one oxidant, and a buffer solution; wherein the enzymatic chlorination is followed by coupling formula (II), a salt thereof, or a hydrate thereof, with a compound of formula (IV):

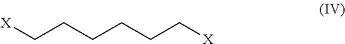

the coupling further comprising one or more base, and wherein X is a leaving group.

In an embodiment, the at least one chloride donor is sodium chloride, the at least one oxidant is hydrogen peroxide, and buffer solution is phosphate buffer.

In an embodiment, the enzymatic chlorination is carried out at a pH of about 2.7.

In an embodiment, the one or more base is sodium hydroxide and X is bromine.

In an embodiment, the coupling of formula (II) and formula (IV) is carried out in butyronitrile at about 100° C.

In an embodiment, a composition comprises the chlorhexidine prepared by any method disclosed herein.

These and other embodiments and features of the disclosure will become more apparent through reference to the following description, the accompanying figures, and the claims. Furthermore, it is to be understood that the features of the various embodiments described herein are not mutually exclusive and can exist in various combinations and permutations.

### DETAILED DESCRIPTION

The present disclosure provides an improved method of preparing chlorhexidine, a pharmaceutically acceptable salt thereof, or a hydrate thereof; the method including an enzymatic chlorination step. The present disclosure avoids the use of PCA, hexamethylenediamine, and dicyanamide as starting materials and therefore results in chlorhexidine, a pharmaceutically acceptable salt thereof, or a hydrate thereof. The present disclosure also provides for a method of preparing chlorhexidine, a pharmaceutically acceptable salt thereof, or a hydrate thereof, where the method includes an enzymatic chlorination step; the method further comprising converting chlorhexidine free base, a pharmaceutically acceptable salt thereof, or a hydrate thereof, into another pharmaceutically acceptable salt thereof, or hydrate thereof.

Throughout this disclosure, various quantities, such as amounts, sizes, dimensions, proportions and the like, are presented in a range format. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “includes”, “comprises”, “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

### I. SYNTHETIC ROUTE

The present disclosure provides for a method of preparing chlorhexidine, a salt thereof, or a hydrate thereof, utilizing an enzymatic chlorination step in conjunction with one or more other steps. In some embodiments, the enzymatic chlorination step is carried out before a coupling step. In an embodiment, the enzymatic chlorination step is carried out sequentially with a coupling step. In other embodiments the enzymatic chlorination step is carried out after the coupling step. In one embodiment, the enzymatic chlorination step and coupling step are carried out without the need for purification between steps. In another embodiment, the enzymatic chlorination step and coupling step are carried out with full or partial purification after each step. In one embodiment, full or partial purification is carried out after each step.

In one embodiment, the method of preparing chlorhexidine comprises an enzymatic chlorination step to create compound of formula (II):

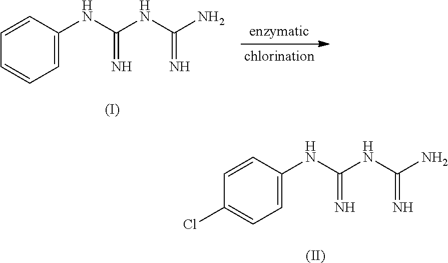

Formula (I) can be phenyl biguanide free base or a salt form thereof, where the salt form may be an inorganic or organic salt. The 1-(4-chlorophenyl)biguanide of formula (II) can be the free base, a hydrate thereof, or a salt thereof, wherein the salt can be an inorganic or organic salt, including but not limited to, a chloride, bromide, iodide, nitrate, phosphate, sulfate, acetate, formate, pivalate, stearate, carbonate, pyruvate, glutamate, oxylate, or urate salt. In one embodiment formula (II) is a bromide, acetate, or chloride salt. In another embodiment formula (II) is a chloride salt.

The enzymatic chlorination is carried out using chloroperoxidase (CPO), wherein CPO may be derived from the organism Caldariomyces fumago, may be chemically modified including but not limited to reaction with an anhydride, or coated on a scaffold including but not limited to a nanoparticle. In an embodiment, CPO can be used in a concentration of about 1 U/mL to about 30 U/mL. In another embodiment, CPO is used in a concentration of about 5 U/mL to about 20 U/mL. In an embodiment, CPO is used in a concentration of about 10 U/mL to about 20 U/mL. In an embodiment, CPO is used in a concentration of about 20 U/mL. In an embodiment, CPO may be introduced to the reaction in a single aliquot, or portion wise over a period of time.

The enzymatic chlorination further comprises one or more oxidant, one or more chloride donor, and a buffer solution. The enzymatic chlorination may further optionally comprise a cofactor or co-solvent.

The oxidant can be selected from, but is not limited to, hydrogen peroxide, dibenzoyl peroxide, peroxycarboxylic acid, calcium peroxide, lithium peroxide or combinations thereof. In an embodiment, the oxidant is hydrogen peroxide. In one embodiment, the oxidant is present in about 1.0 to about 10.0 equivalents relative to formula (I). In another embodiment, the oxidant is present in about 1.0 to about 5.0 equivalents, in about 2.0 to about 4.0 equivalents or about 2.0 to about 3.0 equivalents relative to formula (I). In a further embodiment, the oxidant is present in about 1.0, about 2.0, about 3.0, about 4.0, about 5.0, about 6.0, about 7.0, about 8.0, about 9.0, or about 10.0 equivalents relative to formula (I). In an embodiment, the oxidant is present in about 2.0 equivalents.

The chloride donor can be selected from, but is not limited to, hydrogen chloride, potassium chloride, sodium chloride, lithium chloride, sodium hypochlorite, calcium hypochlorite, chlorine water, chlorine gas, chloramines, chlorine dioxide, and combinations thereof. In one embodiment, the enzymatic chlorination step comprises chloride. In one embodiment the chloride donor is present in about 1.0 to about 20.0 equivalents relative to formula (I). In another embodiment the chloride donor is present in about 1.0 to about 10.0 equivalents relative to formula (I). In yet another embodiment the chloride donor is present in about 1.0 to about 5.0 equivalents relative to formula (I). In an embodiment, the chloride donor is present in about 1.0, about 2.0, about 3.0, about 4.0, about 5.0, about 6.0, about 7.0, about 8.0, about 9.0, about 10.0, about 11.0, about 12.0, about 13.0, about 14.0, about 15.0, about 16.0, about 17.0, about 18.0, about 19.0, or about 20.0 equivalents relative to formula (I). In an embodiment, the chloride donor is present in about 2.0 equivalents relative to formula (I).

The buffer solution may be selected from, but is not limited to, a phosphate buffer, acetate buffer, citrate buffer, trifluoroacetic acid buffer, phosphoric acid buffer, formic acid buffer, ammonium formate buffer, ammonium bicarbonate buffer, or borate buffer. The buffer solution may be a mildly acidic buffer solution or a strongly acidic buffer solution. In an embodiment, the buffer solution is a citrate buffer solution or a phosphate buffer solution. In an embodiment, the buffer solution is a phosphate buffer solution.

[INVENTORS: Can you comment on the importance of pH in the process?] In an embodiment the enzymatic chlorination is carried out at a pH of about 1.0 to about 7.0. In another embodiment the enzymatic chlorination is carried out at a pH of about 2.0 to about 5.0, about 2.0 to about 4.0, about 2.0 to about 3.0, about 2.5 to about 3.0, or about 2.7 to about 3.0. In an embodiment the enzymatic chlorination is carried out at a pH of about 2.5 to about 3.0, or about 2.7 to about 3.0. In an embodiment the enzymatic chlorination is carried out at a pH of about 2.5, about 2.7, or about 3.0.

The optional cofactor may be selected from a heme cofactor, vanadate cofactor, a combination thereof, or may be metal-free. In another embodiment, the reaction may optionally contain DMF as a co-solvent. A person skilled in the art would understand that there are other solvents and/or cofactors that can be utilized. These can include, but are not limited to: dichloromethane, chloroform, dimethylformamide, dimethyl sulfoxide, benzene, toluene, trifluorotoluene, hexane, heptane, petroleum ether, dimethyl ether, tetrahydrofuran, acetone, ethyl acetate, dioxane, ethanol, tert-butanol, ethoxyethanol, acetonitrile, proprionitrile, butyronitrile, or a combination thereof.

[INVENTORS: Can you comment on the importance of temperature in the process?] In an embodiment the enzymatic chlorination is carried out at a temperature of about 10° C. to about 60° C., about 20° C. to about 40° C., or about 20° C. to about 30° C. In another embodiment the enzymatic chlorination is carried out at a temperature of about 10° C., about 15° C., about 20° C., about 25° C., about 30° C., about 35° C., about 40° C., about 45° C., about 50° C., or about 55° C. In still another embodiment the enzymatic chlorination is carried out at room temperature. In one embodiment, the enzymatic chlorination is carried out at 25° C.

In an embodiment, the enzymatic chlorination produces less than about 5%, less than about 2%, or less than about 1% of 1-(3-chlorophenyl)biguanide. In another embodiment, the enzymatic chlorination produces a ratio of 1-(4-chlorophenyl)biguanide to 1-(2-chlorophenyl)biguanide of greater than 2:1 in favor of 1-(4-chlorophenyl)biguanide. In still another embodiment, the enzymatic chlorination produces a ratio of 1-(4-chlorophenyl)biguanide to 1-(2-chlorophenyl)biguanide of greater than 3:1 in favor of 1-(4-chlorophenyl)biguanide. In an embodiment, the enzymatic chlorination produces a ratio of 1-(4-chlorophenyl)biguanide to 1-(2-chlorophenyl)biguanide of greater than 4:1 in favor of 1-(4-chlorophenyl)biguanide. In an embodiment, the enzymatic chlorination produces a ratio of 1-(4-chlorophenyl)biguanide to 1-(2-chlorophenyl)biguanide of greater than 5:1 in favor of 1-(4-chlorophenyl)biguanide. In an embodiment, the enzymatic chlorination produces less than about 5%, less than about 3%, or less than about 1% of a dichlorinated compound where two or more chlorine atoms are installed on the aromatic ring. In an embodiment, the yield of formula (II) is at least 30%, at least 40%, at least 50%, at least, 60%, at least 70%, at least 80%, or at least 90%. In an embodiment, the yield of formula (II) is greater than 50%. In an embodiment, the yield of formula (II) is greater than 65%. In an embodiment, formula (II), a salt thereof, or a hydrate thereof has a purity of greater than about 90%. In an embodiment, formula (II), a salt thereof, or a hydrate thereof has a purity of greater than about 95%. In an embodiment, formula (II), a salt thereof, or a hydrate thereof has a purity of greater than about 98%. In an embodiment, formula (II), a salt thereof, or a hydrate thereof has a purity of greater than about 99.5%.

In some embodiments, the CPO can be separated from the reaction mixture by filtration, centrifuge, precipitation, chromatography, affinity chromatography, solvent phase separation, washing, extraction, or a combination thereof. In another embodiment, the CPO is further purified through any conventional means known to a person of skill in the art before reuse. In an embodiment, the recovered CPO can be reused in a chlorination reaction. In an embodiment, the recovered CPO can be reused by itself, or in combination with new CPO. In some embodiments, the CPO can be immobilized using a natural matrix, a natural support, synthetic matrix or synthetic support. The immobilization method is selected from an adsorption method, covalent method, entrapment method, cross-linking method, membrane confinement method, or affinity immobilization method. The immobilization matrix or support may be selected from, but is not limited to, an alginate, collagen, cellulose, microcrystalline cellulose, K-carrageenan, chitosan, chitin, gelatin, starch, pectin, sepharose, zeolites, ceramics, celite, silica, glass, activated carbon, charcoal, multifunctional glutaraldehyde, bifunctional glutaraldehyde, bisdiazobenzidine and hexamethylene diisocyanate, coconut fibers, acetylized kaolin, silanized molecular sieves, octyl-agarose, octadecyl-sepabeads, 3-hydroxybutyrate-co-hydroxyvalerate, 1,4-butenediol diglycidyl ether-activated byssus threads, mesoporous silica, mesoporous silica nanoparticles, cyanogen bromide (CNBr)-agarose and CNBr-activated-sepharose, alginate-gelatin-calcium hybrid carrier, polypropylene-based hydrophobic granules, and polysaccharide derivatives. In an embodiment the CPO can be chemically modified by reaction with, for example, an anhydride, a carbodiimide, or periodate oxidation.

In one embodiment, the method of preparing chlorhexidine comprises an enzymatic chlorination of formula (III):

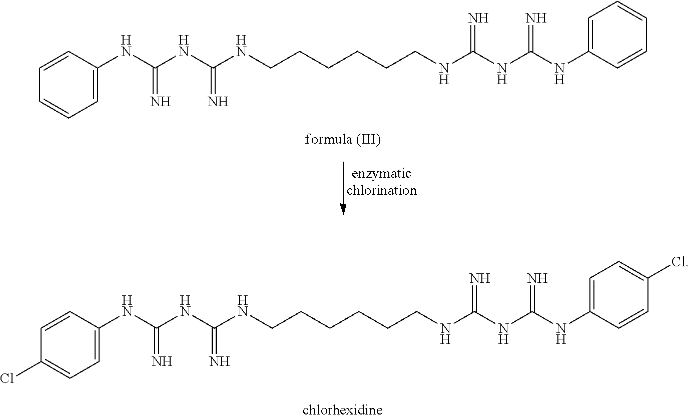 [SMILES: N=C(NCCCCCCNC(=N)NC(=N)Nc1ccccc1)NC(=N)Nc1ccccc1 ; N=C(NCCCCCCNC(=N)NC(=N)Nc1ccc(Cl)cc1)NC(=N)Nc1ccc(Cl)cc1]

Formula (III) can be used as a free base or a salt form thereof, where the salt form may be an inorganic or organic salt. The chlorhexidine can be obtained as the free base, converted to a salt thereof, or obtained from the reaction as a salt thereof; wherein the salt can be an inorganic or organic salt, including but not limited to, a chloride, bromide, iodide, nitrate, phosphate, sulfate, acetate, formate, pivalate, stearate, carbonate, pyruvate, glutamate, oxylate, or urate salt. In some embodiments, chlorhexidine prepared from formula (III) may be further converted into a salt or hydrate. In some embodiments, the chlorhexidine prepared from formula (III) is converted into a salt selected from a monoacetate salt, a diacetate salt, a monohydrochloride salt, a dihydrochloride salt, a monogluconate salt, or a digluconate salt. In one embodiment, chlorhexidine prepared from formula (III) is converted into chlorohexidine gluconate.

The enzymatic chlorination to form chlorhexidine comprises CPO, one or more oxidant, on or more chloride donor, and a buffer solution; wherein the amounts, ratios, and identity of each element have been described above for the formation of formula (II). The enzymatic chlorination to form chlorhexidine may further comprise a pH and temperature as described above for the formation of formula (II). The enzymatic chlorination to form chlorhexidine may further optionally comprise a cofactor or co-solvent as described above for the formation of formula (II).

In an embodiment, the enzymatic chlorination to form chlorhexidine produces less than about 5%, less than about 2%, or less than about 1% of meta-substituted chlorinated compounds. In another embodiment, the enzymatic chlorination produces a ratio of para-substitution to ortho-substitution of greater than 2:1 in favor of para-substitution. In still another embodiment, the enzymatic chlorination produces a ratio of para-substitution to ortho-substitution of greater than 3:1 in favor of para-substitution. In an embodiment, the enzymatic chlorination produces a ratio of para-substitution to ortho-substitution of greater than 4:1 in favor of para-substitution. In an embodiment, the enzymatic chlorination produces a ratio of para-substitution to ortho-substitution of greater than 5:1 in favor of para-substitution. In an embodiment, the enzymatic chlorination produces less than about 5%, less than about 3%, or less than about 1% of an overchlorinated compound. In an embodiment, the yield of chlorhexidine is at least 20%, at least 30%, at least 40%, at least 50%, at least, 60%, at least 70%, at least 80%, or at least 90%. In an embodiment, the yield of chlorhexidine is greater than 50%. In an embodiment, the yield of chlorhexidine is greater than 65%. In an embodiment, chlorhexidine, a salt thereof, or a hydrate thereof has a purity of greater than about 90%. In an embodiment, chlorhexidine, a salt thereof, or a hydrate thereof has a purity of greater than about 95%. In an embodiment, chlorhexidine, a salt thereof, or a hydrate thereof has a purity of greater than about 98%. In an embodiment, chlorhexidine, a salt thereof, or a hydrate thereof has a purity of greater than about 99.5%.

In one embodiment, the method of preparing chlorhexidine comprises a coupling step using formula (II) and formula (IV):

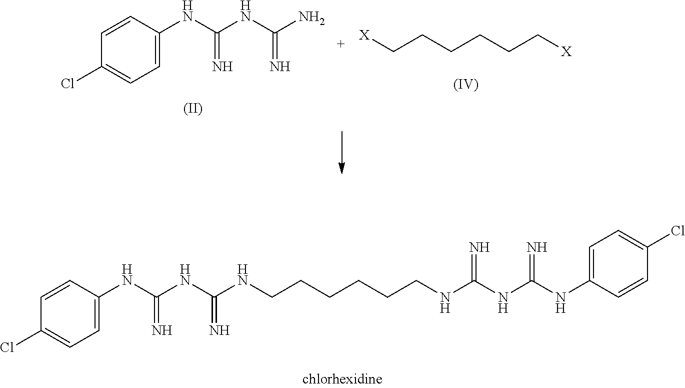

In an embodiment, the 1-(4-chlorophenyl)biguanide of formula (II) can be the free base, a hydrate thereof, or a salt thereof; wherein the salt can be an inorganic or organic salt, including but not limited to, a chloride, bromide, iodide, nitrate, phosphate, sulfate, acetate, formate, pivalate, stearate, carbonate, pyruvate, glutamate, oxylate, or urate salt. In one embodiment formula (II) is a bromide, acetate, or chloride salt. In another embodiment formula (II) is a chloride salt.

In an embodiment, X of formula (IV) is selected from the group consisting of hydroxyl, bromine, chlorine, iodine, tosylate, mesylate, triflate, nosylate, substituted ammonium, or other suitable leaving group. In an embodiment, X is bromine.

In an embodiment, the coupling step can be carried out using a molar ratio in the range of about 100:1 to about 2:1 of formula (II) and formula (IV), respectively. In another embodiment, the coupling step can be carried out using a molar ratio in the range of about 50:1 to about 2:1 of formula (II) and formula (IV), respectively. In yet another embodiment, the coupling step can be carried out using a molar ratio in the range of about 10:1 to about 2:1 of formula (II) and formula (IV), respectively. In still another embodiment, the coupling step can be carried out using a molar ratio in the range of about 5:1 to about 2:1 of formula (II) and formula (IV), respectively. In an embodiment, the coupling step can be carried out using a molar ratio of about 2:1 of formula (II) and formula (IV), respectively. In an embodiment, the coupling step is carried out where formula (II) and formula (IV) are added together at once, dropwise, in incremental portions, or by continuous flow.

In an embodiment, the coupling step further comprises one or more base. In an embodiment, the coupling step may comprise one or more solvent or be conducted neat in melted substrate. In another embodiment, the coupling step may optionally comprise a one or more additive such as a catalyst, proton sponge, molecular sieves, phase transfer reagent, or combinations thereof.

In an embodiment, the one or more base is selected from an amine base, alkoxide base, or inorganic base. In another embodiment, the one or more base is selected from, but not limited to, sodium hydroxide, sodium methoxide, sodium ethoxide, potassium hydroxide, potassium tert-butoxide, triethylamine, ethyldiisopropylamine, 1,8-diazabicyclo[5.4.0]undec-7-ene, 7-methyl-1,5,7-triazabicyclo(4.4.0)dec-5-ene, 1,1,3,3-tetramethylguanidine, collidine, 2,6-lutidine, or combinations thereof. In an embodiment the one or more base is selected from potassium tert-butoxide, sodium hydroxide, or combinations thereof. In an embodiment, the base is selected from sodium hydroxide. In an embodiment, the one or more base is used in about 1.0 to about 20.0 equivalents relative to formula (IV). In another embodiment, the one or more base is used in about 2.0 to about 15.0 equivalents relative to formula (IV). In an embodiment, the one or more base is used in about 2.0 to about 4.0 equivalents relative to formula (IV). In an embodiment, the one or more base is used in about 10.0 to about 12.0 equivalents relative to formula (IV). In an embodiment, the one or more base is used in about 2.0 equivalents relative to formula (IV). In an embodiment, the one or more base is used in about 11.0 equivalents relative to formula (IV).

In one embodiment, the coupling step is carried out in a solvent selected from, but not limited to, dichloromethane, chloroform, dimethylformamide, dimethyl sulfoxide, benzene, toluene, trifluorotoluene, hexane, heptane, petroleum ether, dimethyl ether, tetrahydrofuran, acetone, ethyl acetate, dioxane, ethanol, tert-butanol, ethoxyethanol, acetonitrile, proprionitrile, butyronitrile, or a combination thereof. In one embodiment, the coupling step is carried out in a solvent selected from dimethylformamide, dimethyl sulfoxide, toluene, acetonitrile, proprionitrile, butyronitrile, or combinations thereof. In an embodiment, the coupling step is carried out in butyronitrile.

In an embodiment, the coupling step is carried out at a temperature from about 0° C. to about 150° C. In an embodiment, the coupling step is carried out at a temperature from about 20° C. to about 40° C. or about 90° C. to about 120° C. In an embodiment, the coupling step is carried out at ambient temperature, 20° C., or 25° C. In an embodiment, the coupling step is carried out at about 100° C. In an embodiment, the reaction may be conducted at pressures above atmospheric pressure, including but not limited to, a sealed vessel.

In an embodiment, the coupling step may further comprise one or more additive such as a catalyst, proton sponge, molecular sieves, phase transfer reagent, or combinations thereof. In an embodiment, the one or more additive is selected from, but is not limited to, sodium iodide, potassium iodide, ammonium iodide, 1,8-bis(dimethylamino) naphthalene, 2,2,6,6-Tetramethylpiperidine, molecular sieves, a basic resin, tetramethylethylenediamine, tetrabutylammonium bromide, or combinations thereof.

In an embodiment, the chlorhexidine can be obtained as the free base or a salt thereof; wherein the salt can be an inorganic or organic salt, including but not limited to, a chloride, bromide, iodide, nitrate, phosphate, sulfate, acetate, formate, pivalate, stearate, carbonate, pyruvate, glutamate, oxylate, or urate salt. In some embodiments, chlorhexidine prepared from formula (II) and formula (IV) may be further converted into a salt or hydrate. In some embodiments, the chlorhexidine prepared from formula (II) and formula (IV) is converted into a salt selected from a monoacetate salt, a diacetate salt, a monohydrochloride salt, a dihydrochloride salt, a monogluconate salt, or a digluconate salt. In one embodiment, chlorhexidine prepared from formula (II) and formula (IV) is converted into chlorohexidine gluconate.

In an embodiment, the chlorhexidine prepared from formula (II) and formula (IV) is obtained in a yield of at least 30%, at least 40%, at least 50%, at least, 60%, at least 70%, at least 80%, or at least 90%. In an embodiment, the yield of chlorhexidine prepared from formula (II) and formula (IV) is greater than 50%. In an embodiment, the yield of chlorhexidine prepared from formula (II) and formula (IV) is greater than 60%. In an embodiment, the yield of chlorhexidine prepared from formula (II) and formula (IV) is greater than 70%. In an embodiment, the chlorhexidine prepared from formula (II) and formula (IV), a salt thereof, or a hydrate thereof has a purity of greater than about 90%. In an embodiment, the chlorhexidine prepared from formula (II) and formula (IV), a salt thereof, or a hydrate thereof has a purity of greater than about 95%. In an embodiment, the chlorhexidine prepared from formula (II) and formula (IV), a salt thereof, or a hydrate thereof has a purity of greater than about 98%. In an embodiment, the chlorhexidine prepared from formula (II) and formula (IV), a salt thereof, or a hydrate thereof has a purity of greater than about 99.5%.

In one embodiment, the method of preparing chlorhexidine comprises a coupling step to form formula (III):

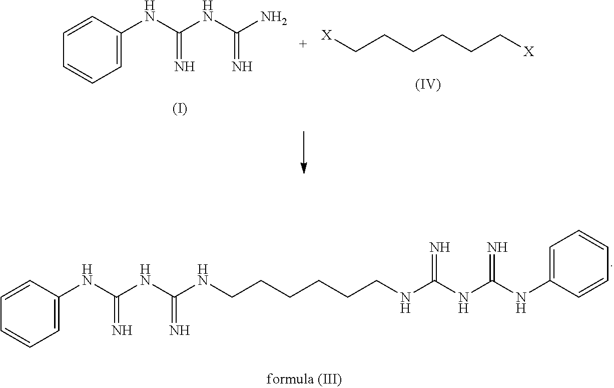

In an embodiment, the phenylbiguanide of formula (I) can be the free base, or a salt thereof; wherein the salt can be an inorganic or organic salt, including but not limited to, a chloride, bromide, iodide, nitrate, phosphate, sulfate, acetate, formate, pivalate, stearate, carbonate, pyruvate, glutamate, oxylate, or urate salt. In one embodiment formula (I) is a bromide, acetate, or chloride salt. In another embodiment formula (I) is a chloride salt.

In an embodiment, the coupling step further comprises one or more base, and one or more solvent. In another embodiment, the coupling step may further optionally comprise a one or more additive such as a catalyst, proton sponge, molecular sieves, phase transfer reagent, or combinations thereof. The identity of X for formula (IV), reactant ratios, rates of addition, the identity of the one or more base and its equivalents, identity of the one or more solvent, the reaction temperature and pressure, and additives are defined similarly as for the coupling step between formulas (II) and (IV), above.

In an embodiment, the chlorhexidine can be obtained as the free base or a salt thereof; wherein the salt can be an inorganic or organic salt, including but not limited to, a chloride, bromide, iodide, nitrate, phosphate, sulfate, acetate, formate, pivalate, stearate, carbonate, pyruvate, glutamate, oxylate, or urate salt. In some embodiments, chlorhexidine prepared from formula (I) and formula (IV) may be further converted into a salt or hydrate.

In an embodiment, formula (III) prepared from formula (I) and formula (IV) is obtained in a yield of at least 30%, at least 40%, at least 50%, at least, 60%, at least 70%, at least 80%, or at least 90%. In an embodiment, the yield of formula (III) prepared from formula (I) and formula (IV) is greater than 50%. In an embodiment, the yield of formula (III) prepared from formula (I) and formula (IV) is greater than 60%. In an embodiment, the yield of formula (III) prepared from formula (I) and formula (IV) is greater than 70%. In an embodiment, formula (III) prepared from formula (I) and formula (IV), a salt thereof, or a hydrate thereof has a purity of greater than about 90%. In an embodiment, formula (III) prepared from formula (I) and formula (IV), a salt thereof, or a hydrate thereof has a purity of greater than about 95%. In an embodiment, formula (III) prepared from formula (I) and formula (IV), a salt thereof, or a hydrate thereof has a purity of greater than about 98%. In an embodiment, formula (III) prepared from formula (I) and formula (IV), a salt thereof, or a hydrate thereof has a purity of greater than about 99.5%.

In an embodiment, chlorhexidine is prepared by a process comprising an enzymatic chlorination step to create the compound of formula (II):

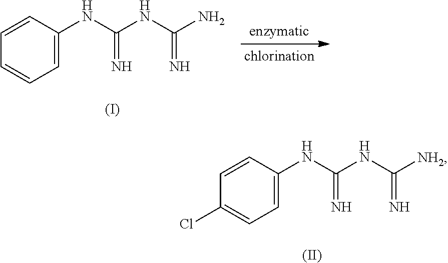

followed by the coupling step:

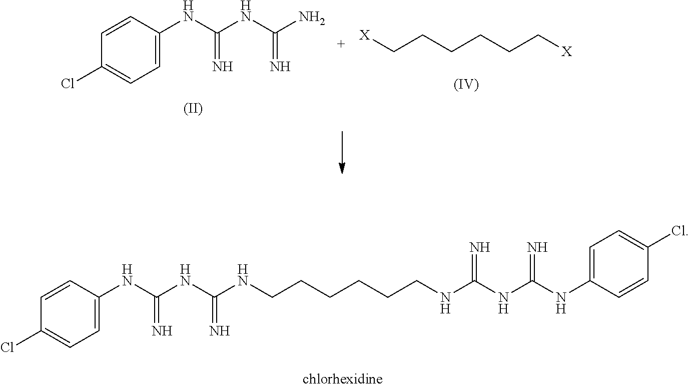

In another embodiment, chlorhexidine is prepared by a process comprising a coupling step:

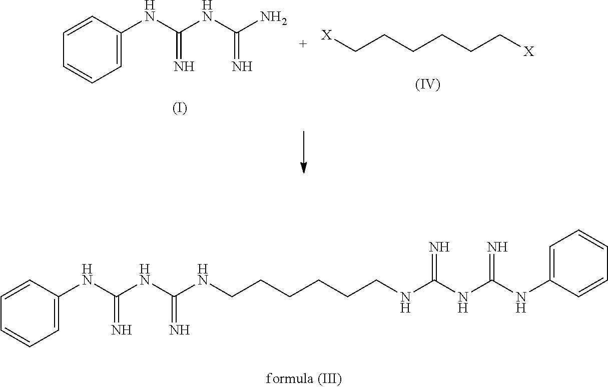

followed by an enzymatic chlorination step:

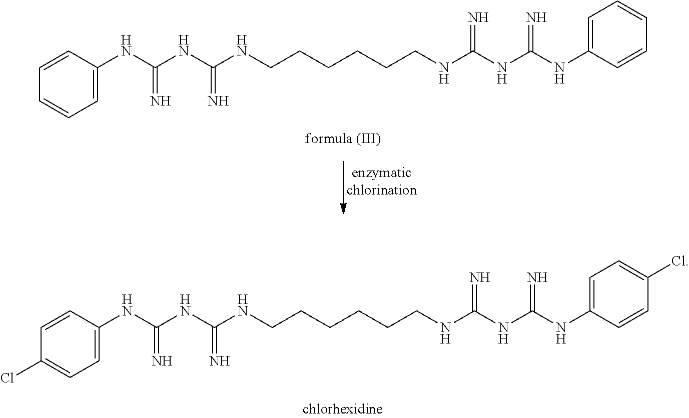 [SMILES: N=C(NCCCCCCNC(=N)NC(=N)Nc1ccccc1)NC(=N)Nc1ccccc1 ; N=C(NCCCCCCNC(=N)NC(=N)Nc1ccc(Cl)cc1)NC(=N)Nc1ccc(Cl)cc1]

In the multi-step sequences, formulas (I), (II), (III), (IV), and chlorhexidine each carry their definitions as described, above, and may be their free base, a salt thereof or a hydrate thereof. Reaction conditions, yields and purities for each step carry the same definitions and identities as described, above. In an embodiment, each multistep sequence independently can produce chlorhexidine in an overall yield of about 40% or greater. In another embodiment, each multistep sequence independently can produce chlorhexidine in an overall yield of about 50% or greater. In yet another embodiment, each multistep sequence independently can produce chlorhexidine in an overall yield of about 70% or greater. In still another embodiment, each multistep sequence independently can produce chlorhexidine in an overall yield of about 90% or greater.

Any of formulas (II), (III), (IV), and chlorhexidine, their salts or hydrates, prepared by any of the embodiments disclosed, above, can be further purified. In an embodiment, purification can be carried out by any conventional method known to a person of skill in the art, and may carried on intermediates, final products, salts or hydrates prepared from intermediates or final products, or a combination thereof. In some embodiments, formula (II), formula (III), or chlorhexidine is further purified using scavenging, absorption, resins, extraction, washing, chromatography, precipitation, recrystallization, filtration, or a combination thereof. In an embodiment, chlorhexidine, a salt thereof, or a hydrate thereof prepared by any of the embodiments disclosed, above, contains no measurable PCA. In an embodiment, chlorhexidine, a salt thereof, or a hydrate thereof prepared by any of the embodiments disclosed, above, contains a negligible amount of PCA, where the PCA does not originate from using PCA as a starting material. In an embodiment, chlorhexidine, a salt thereof, or a hydrate thereof prepared by any of the embodiments disclosed, above, contains PCA in an amount of about 100 ppm or less, where the PCA does not originate from using PCA as a starting material. In another embodiment, chlorhexidine, a salt thereof, or a hydrate thereof prepared by any of the embodiments disclosed, above, contains PCA in an amount of about 50 ppm or less, where the PCA does not originate from using PCA as a starting material.

### II. COMPOSITIONS COMPRISING CHLORHEXIDINE, SALTS THEREOF, OR HYDRATES THEREOF, PREPARED BY THE METHODS DESCRIBED, ABOVE

Chlorhexidine, a salt thereof, or a hydrate thereof prepared by any of the embodiments disclosed above can be used in antibiotic compositions. In some embodiments, a composition comprising chlorhexidine, a salt thereof, or a hydrate thereof prepared by any of the embodiments disclosed above, is an alcohol containing composition or an alcohol-free composition.

In one embodiment, an alcohol-free composition comprises chlorhexidine, a salt thereof, or a hydrate thereof prepared by any of the embodiments disclosed above, wherein the concentration of chlorhexidine, a salt thereof, or a hydrate thereof is about 1% to about 10% weight/weight ratio, preferably about 2% to about 6% weight/weight ratio, and most preferably about 4% weight/weight ratio in a water-based matrix. In one embodiment, an alcohol-free composition comprising chlorhexidine, a salt thereof, or a hydrate thereof prepared by any of the embodiments disclosed above, further comprises a silicone polymer and glycerin.

In some embodiments, an alcohol free composition comprising chlorhexidine, a salt thereof, or a hydrate thereof prepared by any of the embodiments disclosed above, further comprises a silicone polymer wherein the silicone polymer is selected from one or more of: polydimethylsiloxane polymer (Dow Corning 225 Silicone Fluid), dimethiconol fluid in dimethicone (Dow Corning 1403 Silicone Fluid), cyclomethicone and dimethicone copolyl (Dow Corning 3225C Silicone Fluid), silicone glycol (BASF 1066 DCG polyol), and most preferably, cyclopentasiloxane-C30-45 alkyl cetearyl dimethicone crosspolymer (Velvesil™). The silicone polymer may be employed to about 5% weight/weight ratio of the composition, preferably less than 0.1% weight/weight ratio, and most preferably 0.01% weight/weight ratio in a water-based matrix.

In some embodiments, an alcohol-free composition comprising chlorhexidine, a salt thereof, or a hydrate thereof prepared by any of the embodiments disclosed above, further comprises glycerin, wherein is selected from the group consisting of glycerin, propylene glycol, glyceraldehyde, dihydroxyacetone, 1,3-butylene glycol, 2,3-butylene glycol, erythritol, erythrose, erythrulose, ribose, sorbitol, mannitol, and inositol. Glycerin may be employed to less than 1% weight/weight ratio and most preferably 0.25% weight/weight ratio in a water-based matrix.

In some embodiments, an alcohol free composition comprising chlorhexidine, a salt thereof, or a hydrate thereof prepared by any of the embodiments disclosed above, further comprises other ingredients selected from octadecene-1/maleic anhydride copolymer, organofluorinated modified silicone resins, polyvinyl pyrrolidone, hydrogenated copolymers of styrene and butadiene, film forming copolymers, copolymers of eicosene and vinyl pyrrolidone, copolymer of hexadecane and vinyl pyrrolidone, cellulosic ethers, 2-hydroxyethyl methacrylate homopolymer, cocodimethyl ammonium salt of hydrolyzate of wheat protein, guar hydroxy propyltrimonium chloride, hydroxypropylcellulose, lauryldimonium hydroxypropyl hydrolyzed collagen, lauryldimonium hydroxypropyl hydrolyzed wheat protein, polyquaternium-24 and hyaluronic acid, soluble reticulin and soluble wheat protein, stearyldimonium hydroxypropyl hydrolyzed collagen, acrylic/acrylate copolymers, phenoxy dimethicone, dimethicone copolyol phosphate, polydecene/polybutene copolymer, polymethacrylamidopropyl trimonium chloride, polymethylalkyl siloxane, and the like.

In some embodiments, a composition comprising chlorhexidine, a salt thereof, or a hydrate thereof prepared by any of the embodiments disclosed above, is an alcohol containing composition. In some embodiments the alcohol containing composition further comprises a water miscible alcohol and water. In further embodiments, the alcohol containing composition can be a liquid, ointment, lotion, foamable or gel composition.

In some embodiments, the alcohol containing composition comprising chlorhexidine, a salt thereof, or a hydrate thereof prepared by any of the embodiments disclosed above, comprises chlorhexidine, a salt thereof, or a hydrate thereof having a concentration of about 0.001% to about 10% weight/weight ratio in the composition. In another embodiment, the alcohol containing composition comprising chlorhexidine, a salt thereof, or a hydrate thereof prepared by any of the embodiments disclosed above, comprises chlorhexidine, a salt thereof, or a hydrate thereof having a concentration of about 0.1% to about 5% weight/weight ratio in the composition.

In some embodiments, for the alcohol containing composition comprising chlorhexidine, a salt thereof, or a hydrate thereof prepared by any of the embodiments disclosed above, the alcohol is a C₁-C₅ lower alcohol. In some embodiments, the C₁-C₅ lower alcohol is selected from, but not limited to, ethanol, n-propanol, iso-propanol, n-butanol, tert-butanol, iso-butanol, sec-butanol, n-pentanol, or a combination thereof. In some embodiments, the C₁-C₅ lower alcohol is selected from ethanol or iso-propanol. In some embodiments, the C₁-C₅ lower alcohol may be denatured, a commercially available blend, or contain a suitable co-solvent selected from, but not limited to, acetone, hydrocarbons such as isooctane, glycols, ketones, ethers, short chain esters, and combinations thereof. In some embodiments, for the alcohol containing composition comprising chlorhexidine, a salt thereof, or a hydrate thereof prepared by any of the embodiments disclosed above, the alcohol is present in an amount of about 1% to about 90% weight/weight ratio in the composition. In other embodiments, the alcohol is present in an amount of about 5%, about 10%, about 20%, about 35% or about 50% weight/weight ratio in the composition. In some embodiments, for the alcohol containing composition comprising chlorhexidine, a salt thereof, or a hydrate thereof prepared by any of the embodiments disclosed above, the alcohol and water are in a ratio between about 1:100 to about 9:1 alcohol to water. In one embodiment, for the alcohol containing composition comprising chlorhexidine, a salt thereof, or a hydrate thereof prepared by any of the embodiments disclosed above, the alcohol and water are in a ratio between about 1:20 to about 66:34 alcohol to water.

In other embodiments, the alcohol containing composition comprising chlorhexidine, a salt thereof, or a hydrate thereof prepared by any of the embodiments disclosed above further comprises other excipients, where said excipients may be selected from, but are not limited to, other antimicrobials, therapeutic agents, surfactants, carriers, dyes, salts, fragrances, pH adjusters, thickeners, viscosity modifiers, polymers, buffering agents, foam stabilizers, foam enhancers, antioxidants, propellants, conditioning agents, vitamins, chelating agents, emollients, stabilizers, solvents, opacifiers, and similar classes of optional ingredients known to persons skilled in the art.

In some embodiments, the alcohol containing composition comprising chlorhexidine, a salt thereof, or a hydrate thereof prepared by any of the embodiments disclosed above, further comprises one or more surfactants. In some embodiments, the alcohol containing composition comprising chlorhexidine, a salt thereof, or a hydrate thereof prepared by any of the embodiments disclosed above, the surfactant may be selected from, but is not limited to, an anionic surfactant, a cationic surfactant, a nonionic surfactant, an ampholytic surfactant, or combinations thereof. In some embodiments, the alcohol containing composition comprising chlorhexidine, a salt thereof, or a hydrate thereof prepared by any of the embodiments disclosed above, the surfactant may be selected from, but is not limited to, a C₈-C₁₈ alkyl sulfate, a C₈-C₁₈ fatty acid salt, a C₈-C₁₈ alkyl ether sulfate, a C₁-C₁₈ alkamine oxide, a C₈-C₁₈ alkoyl sarcosinate, a C₈-C₁₈ sulfoacetate, a C₈-C₁₈ sulfosuccinate, a C₈-C₁₈ alkyl diphenyl oxide disulfonate, a C₈-C₁₈ alkyl carbonate, a C₈-C₁₈ alphaolefin sulfonate, a methyl ester sulfonate, and mixtures thereof. In some embodiments, the alcohol containing composition comprising chlorhexidine, a salt thereof, or a hydrate thereof prepared by any of the embodiments disclosed above, specific surfactants may include, but is not limited to, lauryl sulfates, octyl sulfates, 2-ethylhexyl sulfates, lauramine oxide, decyl sulfates, tridecyl sulfates, cocoates, lauroyl sarcosinates, lauryl sulfosuccinates, linear C₁₀-diphenyl oxide disulfonates, lauryl sulfosuccinates, lauryl ether sulfates, myristyl sulfates, oleates, stearates, tallates, cocamine oxide, decylamine oxide, myristamine oxide, ricinoleates, cetyl sulfates, and similar surfactants. Additional examples of surfactants can be found in “Handbook of Pharmaceutical Excipients, 6th Ed.,” Rowe, R. C., et al., ed., Pharmaceutical Press, Grayslake, IL (2009) (hereafter Handbook of Pharmaceutical Excipients, 6th Ed.) In some embodiments, the alcohol containing composition comprising chlorhexidine, a salt thereof, or a hydrate thereof prepared by any of the embodiments disclosed above, the surfactants may optionally be combined with a hydrotrope.

In some embodiments, the alcohol containing composition comprising formula (I), or a salt thereof, prepared by any of the embodiments disclosed above, further comprises one or more carriers. In some embodiments, the alcohol containing composition comprising chlorhexidine, a salt thereof, or a hydrate thereof prepared by any of the embodiments disclosed above, the carriers may be selected from, but is not limited to, gelatin, acacia, guar gum, maltodextrin, lactose, polyethylene glycol, sorbitol, or combinations thereof. Additional examples of carriers can be found in Handbook of Pharmaceutical Excipients, 6th Ed.

In some embodiments, the alcohol containing composition comprising chlorhexidine, a salt thereof, or a hydrate thereof prepared by any of the embodiments disclosed above, further comprises one or more chelating agents. In some embodiments, the alcohol containing composition comprising chlorhexidine, a salt thereof, or a hydrate thereof prepared by any of the embodiments disclosed above, the chelating agent may be selected from, but is not limited to, phosphates, citric acid monohydrate, disodium edetate, malic acid, maltol, and combinations thereof. Additional examples of chelating agents can be found in Handbook of Pharmaceutical Excipients, 6th Ed.

In some embodiments, the alcohol containing composition comprising chlorhexidine, a salt thereof, or a hydrate thereof prepared by any of the embodiments disclosed above, further comprises one or more foam enhancers or foam stabilizers. In some embodiments, the alcohol containing composition comprising chlorhexidine, a salt thereof, or a hydrate thereof prepared by any of the embodiments disclosed above, foam enhancers or foam stabilizers may be selected from, but are not limited to, cocamide monoethanolamine, cocamide diethanolamine, soyamide diethanolamine, lauramide diethanolamine, oleamide monoisopropanolamine, stearamide monoethanolamine, myristamide monoethanolamine, lauramide monoethanolamine, capramide diethanolamine, ricinoleamide diethanolamine, myristamide diethanolamine, stearamide diethanolamine, oleylamide diethanolamine, tallowamide diethanolamine, lauramide monoisopropanolamine, tallowamide monoethanolamine, isostearamide diethanolamine, isostearamide monoethanolamine, or combinations thereof. Additional examples of foam enhancers or foam stabilizers can be found in Handbook of Pharmaceutical Excipients, 6th Ed.

In some embodiments, the alcohol containing composition comprising chlorhexidine, a salt thereof, or a hydrate thereof prepared by any of the embodiments disclosed above, further comprises one or more buffering agents. In some embodiments, the alcohol containing composition comprising chlorhexidine, a salt thereof, or a hydrate thereof prepared by any of the embodiments disclosed above, the buffering agent may be selected from, but is not limited to, inorganic phosphates, sulfates, carbonates, dioic acids, organic acids, or combinations thereof. Additional examples of buffering agents can be found in Handbook of Pharmaceutical Excipients, 6^{th} Ed.

In some embodiments, the alcohol containing composition comprising chlorhexidine, a salt thereof, or a hydrate thereof prepared by any of the embodiments disclosed above, further comprises one or more pH adjuster. In some embodiments, the alcohol containing composition comprising chlorhexidine, a salt thereof, or a hydrate thereof prepared by any of the embodiments disclosed above, the pH adjuster may be selected from an acid and bases. In some embodiments, the alcohol containing composition comprising chlorhexidine, a salt thereof, or a hydrate thereof prepared by any of the embodiments disclosed above, the basic pH adjuster may be selected from, but is not limited to, ammonia; sodium, potassium, and lithium hydroxide; monoethanolamine; triethylamine; isopropanolamine; diethanolamine; triethanolamine; sodium and potassium bicarbonate; sodium and potassium carbonate; or combinations thereof. In some embodiments, the alcohol containing composition comprising chlorhexidine, a salt thereof, or a hydrate thereof prepared by any of the embodiments disclosed above, the acidic pH adjuster may be selected from, but is not limited to, mineral acids such as hydrochloric acid, nitric acid, phosphoric acid, and sulfuric acid; organic acids such as citric acid, glycolic acid, lactic acid and acetic acid; or combinations thereof. The identity of the pH adjusters is not limited and any pH adjuster known in the art, alone or in combination, can be used.

In some embodiments, the alcohol containing composition comprising chlorhexidine, a salt thereof, or a hydrate thereof prepared by any of the embodiments disclosed above, further comprises one or more solvents. In some embodiments, the alcohol containing composition comprising chlorhexidine, a salt thereof, or a hydrate thereof prepared by any of the embodiments disclosed above, the solvent may be selected from, but is not limited to, acetone, dimethylisosorbide, isooctane, lower alcohols, acetone, methyl ethyl ketone, volatile ethers, water, toluene, or combinations thereof.

In addition to the aspects and embodiments described and provided elsewhere in the present disclosure, the embodiments of the following non-limiting list are also contemplated.
1. A method of preparing the compound of formula (II),

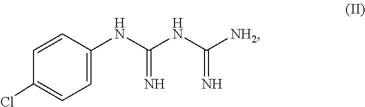 [SMILES: N=C(N)NC(=N)Nc1ccc(Cl)cc1]

a salt thereof, or hydrate thereof; the method comprising enzymatic chlorination of formula (I):

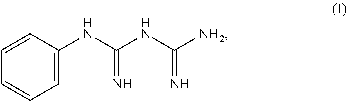 [SMILES: N=C(N)NC(=N)Nc1ccccc1]

or a salt thereof; wherein the chlorination is carried out with chloroperoxidase (CPO), at least one chloride donor, at least one oxidant, and a buffer solution.
2. The method of clause 1, wherein the at least one chloride donor is selected from hydrogen chloride, potassium chloride, sodium chloride, lithium chloride, sodium hypochlorite, calcium hypochlorite, chlorine water, chlorine gas, chloramines, chlorine dioxide, or combinations thereof.
3. The method of clause 1 or clause 2, wherein the at least one chloride donor is sodium chloride.
4. The method of any one of clauses 1 to 3, wherein the at least one oxidant is selected from hydrogen peroxide, dibenzoyl peroxide, peroxycarboxylic acid, calcium peroxide, lithium peroxide or combinations there.
5. The method of any one of clauses 1 to 4, wherein the at least one oxidant is hydrogen peroxide.
6. The method of any one of clauses 1 to 5, wherein the buffer is a phosphate buffer.
7. The method of any one of clauses 1 to 6, wherein the pH is from about 2.7.
8. The method of any one of clauses 1 to 7, wherein formula (II) is produced in greater than about a 50% yield.
9. A method of preparing chlorhexidine, a salt thereof, or a hydrate thereof, comprising combining a compound of formula (II):

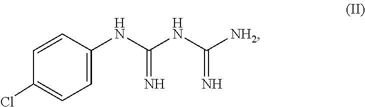 [SMILES: N=C(N)NC(=N)Nc1ccc(Cl)cc1]

a salt thereof, or a hydrate thereof, with a compound of formula (IV):

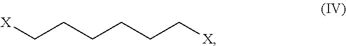

in further combination with one or more base, and wherein X is a leaving group.
10. The method of clause 9, wherein formula (II) is the hydrochloride salt.
11. The method of clause 9 or clause 10, wherein X is selected from hydroxyl, bromine, chlorine, iodine, tosylate, mesylate, triflate, nosylate, or substituted ammonium.
12. The method of any one of clauses 9 to 11, wherein X is bromine.
13. The method of any one of clauses 9 to 12, wherein the one or more base is selected from sodium hydroxide, sodium methoxide, sodium ethoxide, potassium hydroxide, potassium tert-butoxide, triethylamine, ethyldiisopropylamine, 1,8-diazabicyclo[5.4.0]undec-7-ene, 7-methyl-1,5,7-triazabicyclo(4.4.0)dec-5-ene, 1,1,3,3-tetramethylguanidine, collidine, 2,6-lutidine, or combinations thereof.
14. The method of any one of clauses 9 to 13, wherein the one or more base is sodium hydroxide.
15. The method of any one of clauses 9 to 14, wherein the reaction is conducted in a solvent, the solvent comprising butyronitrile.
16. The method of any one of clauses 9 to 15, wherein the reaction is carried out at about 100° C.
17. The method of any one of clauses 9 to 16, wherein the yield of chlorhexidine is greater than about 50%.
18. A method of preparing chlorhexidine comprising, the enzymatic chlorination of formula (I):

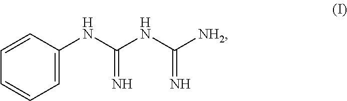 [SMILES: N=C(N)NC(=N)Nc1ccccc1]

or a salt thereof, to form formula (II):

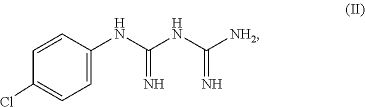 [SMILES: N=C(N)NC(=N)Nc1ccc(Cl)cc1]

a salt thereof, or a hydrate thereof;
the enzymatic chlorination comprising chloroperoxidase (CPO), at least one chloride donor, at least one oxidant, and a buffer solution; wherein the enzymatic chlorination is followed by coupling formula (II), a salt thereof, or a hydrate thereof, with a compound of formula (IV):

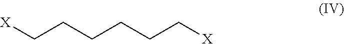

the coupling further comprising one or more base, and wherein X is a leaving group.
19. The method of clause 18, wherein the at least one chloride donor is sodium chloride, the at least one oxidant is hydrogen peroxide, and buffer solution is phosphate buffer.
20. The method of clause 18 or clause 19, wherein the enzymatic chlorination is carried out at a pH of about 2.7.
21. The method of any one of clauses 18 to 20, wherein the one or more base is sodium hydroxide and X is bromine.
22. The method of any one of clauses 18 to 21, where in the coupling of formula (II) and formula (IV) is carried out in butyronitrile at about 100° C.
23. A composition comprising chlorhexidine, wherein the chlorhexidine is prepared by the method of any one of clauses 1 to 8.
24. A composition comprising chlorhexidine, wherein the chlorhexidine is prepared by the method of any one of clauses 9 to 17.
25. A composition comprising chlorhexidine, wherein the chlorhexidine is prepared by the method of any one of clauses 18 to 22.

### III. EXAMPLES

### Example 1

In an example, phenylbiguanide (50 mg, 1.0 equiv.), sodium chloride (33 mg, 2.0 equiv.), and CPO (0.0337 ml, 1000 units) were added to phosphate buffer (pH 3, 50 ml) at 25° C. with stirring. Reaction was initiated by addition of hydrogen peroxide (2.0 equiv.) and the reaction stirred for 24 h. Product was confirmed by mass spectrometry, with the product M+1 peak of 212.

### Example 2

In an example, phenylbiguanide (100 mg, 1.0 equiv.), sodium chloride (66 mg, 2.0 equiv.), and CPO (0.0169 ml, 500 units) were added to phosphate buffer (pH 3, 25 ml) at 25° C. with stirring. Reaction was initiated by addition of hydrogen peroxide (2.0 equiv.) and the reaction stirred for 6 h. Product was confirmed by mass spectrometry, with the product M+1 peak of 212.

### Example 3

In an example, phenylbiguanide (100 mg, 1.0 equiv.), sodium chloride (66 mg, 2.0 equiv.), and hydrogen peroxide (2.0 equiv.) were added to phosphate buffer (pH 2.7, 50 ml) at 25° C. CPO (0.0337 ml, 1000 units) was added to 1 mL of phosphate buffer (pH 2.7) and added in 150 μL increments at regular 10-minute intervals. The reaction was monitored by HPLC and after the seventh addition of CPO solution the reaction resulted in a 68% yield of 1-(4-chlorophenyl)biguanide.

### Example 4

In an example, 1-(4-chlorophenyl)biguanide hydrochloride salt (0.426 g, 2.0 equiv.) is combined with dibromohexane (0.16 ml, 1.0 equiv.) and potassium tert-butoxide (0.448 g, 4.0 equiv.) in DMSO at 25° C. The reaction was conducted for 24 hours resulting in chlorhexidine at a 12.15% yield. Chlorhexidine formation is confirmed by LCMS with a peak having a mass of 505.0 and a m/2e peak at 253.1.

### Example 5

In an example, 1-(4-chlorophenyl)biguanide hydrochloride salt (0.426 g, 2.0 equiv.) is combined with dibromohexane (0.16 ml, 1.0 equiv.) and potassium tert-butoxide (0.448 g, 4.0 equiv.) in DMSO at 120° C. The reaction was conducted for 24 hours resulting in chlorhexidine at a 6.8% yield. Chlorhexidine formation is confirmed by LCMS and mass spectrometry.

### Example 6

In an example, 1-(4-chlorophenyl)biguanide hydrochloride salt (0.426 g, 2.0 equiv.) is combined with dibromohexane (0.16 ml, 1.0 equiv.) in DMSO:toluene (1:1) at 25° C. Potassium tert-butoxide (0.448 g, 4.0 equiv.) is added and the reaction conducted for 24 hours resulting in chlorhexidine at a 7.6% yield. Chlorhexidine formation is confirmed by LCMS and mass spectrometry.

### Example 7

In an example, 1-(4-chlorophenyl)biguanide hydrochloride salt (0.213 g, 1.0 equiv.) is combined with dibromohexane (0.16 ml, 1.0 equiv.) and potassium tert-butoxide (0.6 g, 5.0 equiv.) in THF at 80° C. The reaction was conducted for 24 hours resulting in chlorhexidine at a 12.657% yield. Chlorhexidine formation is confirmed by LCMS and mass spectrometry.

### Example 8

In an example, 1-(4-chlorophenyl)biguanide hydrochloride salt (0.426 g, 2.0 equiv.) was melted by heating at 200° C. along with dibromohexane (0.16 ml, 1.0 equiv.) in chlorhexidine at a 1.85% yield. Chlorhexidine formation is confirmed by LCMS and mass spectrometry.

### Example 9

In an example, 1-(4-chlorophenyl)biguanide hydrochloride salt (0.426 g, 2.0 equiv.) is combined with dibromohexane (0.16 ml, 1.0 equiv.) and sodium hydroxide (0.448 g, 11.0 equiv.) in butyronitrile (10 ml) at 100° C. Chlorhexidine formation is in a yield of 73.9% and product formation was confirmed by LCMS and mass spectometry.

### Example 10

In an example, dibromohexane (0.16 ml, 1.0 equiv.) and potassium iodide (0.332 g, 2.0 equiv.) were added in DMF in a first flask and heated at 100° C. for 1 hour. Phenyl biguanide HCl (0.426 g, 2.0 equiv.) and potassium tertiary butoxide were added in DMF in a second flask at 20° C. for 1 h. The first flask was cooled to 20° C. and added to the second flask. The reaction was stirred overnight at 25° C. Water was added and the mixture extracted with n-butanol, and concentrated to dryness. Product was visible by mass spectrometry, showing a M+1 a characteristic M/2e peak 218.

### Example 11

In an example, deschlorhexidine (70 mg, 1.0 equiv.), sodium chloride (66 mg, 2.0 equiv.), and CPO (0.016 ml, 500 units) were added to phosphate buffer (pH 3, 50 ml) at 25° C. with stirring. Reaction was initiated by addition of hydrogen peroxide (1.0 equiv.) and the reaction stirred for 3 h. The product M+1 peak of 505 was observed.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of preparing the compound of formula (II), 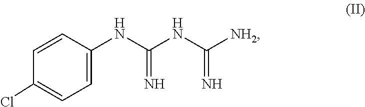 [SMILES: N=C(N)NC(=N)Nc1ccc(Cl)cc1]
a salt thereof, or hydrate thereof; the method comprising enzymatic chlorination of formula (I): 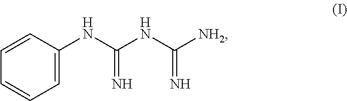 [SMILES: N=C(N)NC(=N)Nc1ccccc1]
or a salt thereof; wherein the chlorination is carried out with chloroperoxidase (CPO), at least one chloride donor, at least one oxidant, and a buffer solution.

2. The method of claim 1, wherein the at least one chloride donor is selected from hydrogen chloride, potassium chloride, sodium chloride, lithium chloride, sodium hypochlorite, calcium hypochlorite, chlorine water, chlorine gas, chloramines, chlorine dioxide, or combinations thereof.

3. The method of claim 1, wherein the at least one chloride donor is sodium chloride.

4. The method of claim 1, wherein the at least one oxidant is selected from hydrogen peroxide, dibenzoyl peroxide, peroxycarboxylic acid, calcium peroxide, lithium peroxide or combinations there.

5. The method of claim 1, wherein the at least one oxidant is hydrogen peroxide.

6. The method of claim 1, wherein the buffer is a phosphate buffer.

7. The method of claim 1, wherein the pH is from about 2.7.

8. The method of claim 1, wherein formula (II) is produced in greater than about a 50% yield.

9. A method of preparing chlorhexidine, a salt thereof, or a hydrate thereof, comprising combining a compound of formula (II): 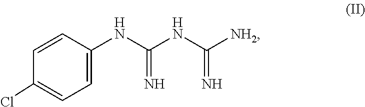 [SMILES: N=C(N)NC(=N)Nc1ccc(Cl)cc1]
a salt thereof, or a hydrate thereof, with a compound of formula (IV): 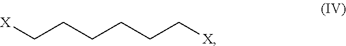
in further combination with one or more base, and wherein X is a leaving group.

10. The method of claim 9, wherein formula (II) is the hydrochloride salt.

11. The method of claim 9, wherein X is selected from hydroxyl, bromine, chlorine, iodine, tosylate, mesylate, triflate, nosylate, or substituted ammonium.

12. The method of claim 9, wherein X is bromine.

13. The method of claim 9, wherein the one or more base is selected from sodium hydroxide, sodium methoxide, sodium ethoxide, potassium hydroxide, potassium tert-butoxide, triethylamine, ethyldiisopropylamine, 1,8-diazabicyclo[5.4.0]undec-7-ene, 7-methyl-1,5,7-triazabicyclo(4.4.0)dec-5-ene, 1,1,3,3-tetramethylguanidine, collidine, 2,6-lutidine, or combinations thereof.

14. The method of claim 9, wherein the one or more base is sodium hydroxide.

15. The method of claim 9, wherein the reaction is conducted in a solvent, the solvent comprising butyronitrile.

16. The method of claim 9, wherein the reaction is carried out at about 100° C.

17. The method of claim 9, wherein the yield of chlorhexidine is greater than about 50%.

18. A method of preparing chlorhexidine comprising, the enzymatic chlorination of formula (I): 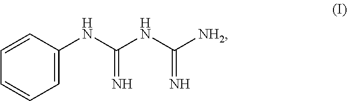 [SMILES: N=C(N)NC(=N)Nc1ccccc1]
or a salt thereof, to form formula (II): 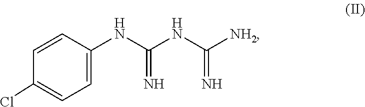 [SMILES: N=C(N)NC(=N)Nc1ccc(Cl)cc1]
a salt thereof, or a hydrate thereof;
the enzymatic chlorination comprising chloroperoxidase (CPO), at least one chloride donor, at least one oxidant, and a buffer solution; wherein the enzymatic chlorination is followed by coupling formula (II), a salt thereof, or a hydrate thereof, with a compound of formula (IV): 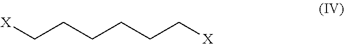
the coupling further comprising one or more base, and wherein X is a leaving group.

19. The method of claim 18, wherein the at least one chloride donor is sodium chloride, the at least one oxidant is hydrogen peroxide, and buffer solution is phosphate buffer.

20. The method of claim 18, wherein the enzymatic chlorination is carried out at a pH of about 2.7.

21. The method of claim 18, wherein the one or more base is sodium hydroxide and X is bromine.

22. The method of claim 18, where in the coupling of formula (II) and formula (IV) is carried out in butyronitrile at about 100° C.

23. A composition comprising chlorhexidine, wherein the chlorhexidine is prepared by the method of claim 1.

24. A composition comprising chlorhexidine, wherein the chlorhexidine is prepared by the method of claim 9.

25. A composition comprising chlorhexidine, wherein the chlorhexidine is prepared by the method of claim 18.
